(19) United States
(12) Patent Application Publication
(10) Pub. No.: US 2024/0207513 A1
(43) Pub. Date: Jun. 27, 2024

(54) AUTOMATIC SYRINGE PUMP NEAR EMPTY ALARM ACTIVATION THRESHOLD PROGRAMMING

(71) Applicant: BAXTER INTERNATIONAL INC., Deerfield, IL (US)

(72) Inventors: Saman Khan, Mumbai (IN); Rajesh Swarnkar, Bangalore (IN); Prasad Digaskar, Mumbai (IN)

(73) Assignee: BAXTER HEALTHCARE SA, Glattpark (Opfikon) (CH)

(21) Appl. No.: 18/392,306

(22) Filed: Dec. 21, 2023

(30) Foreign Application Priority Data
Dec. 27, 2022 (IN) 202241075890

(51) Int. Cl. A61M 5/172

(52) U.S. Cl. CPC A61M 5/172; A61M 2005/1405; A61M 2205/3386; A61M 2205/50; A61M 2205/583

(57) ABSTRACT

Example systems, methods, and apparatus are disclosed herein for the automatic programming of a near empty alarm activation threshold based on an infusion therapy type selected by the user on a syringe pump. The example systems, methods, and apparatus are configured to use a preloaded drug library to determine if the selected infusion therapy type includes a PCA bolus or not, and define an appropriate time- or volume-based alarm activation threshold accordingly. The disclosed systems, methods, and apparatus prevent human programming errors by minimizing the need for extensive user interaction with syringe pumps. Further, the disclosed systems, methods, and apparatus allows for automatic programming to increase efficient infusion administration, and prevent programming errors that might result in patient discomfort or even injury.

### PRIORITY CLAIM AND CROSS-REFERENCE TO RELATED APPLICATIONS

The present application claims priority to Indian Patent Application No. 202241075890, filed on Dec. 27, 2022, and entitled AUTOMATIC SYRINGE PUMP NEAR EMPTY ALARM ACTIVATION THRESHOLD PROGRAMMING, the contents of which are hereby incorporated by reference in their entirety.

### BACKGROUND

Syringe pumps are commonly used for administering pain medication intravenously. Syringe pumps are programmed to deliver an infusion at a continuous constant rate or can include Patient Control Analgesia (PCA) boluses. During an infusion that requires multiple syringes to deliver total volume of drug sequentially, the active syringe will run empty and the user will have to insert a new syringe to continue the infusion. Ordering, verifying, and loading the new syringe may take some time during which the pump is not infusing and the patient will experience discomfort (pain). Currently, pump control system threshold alarms (“syringe near empty alarms”) alert the user before the volume in syringe reaches zero, in order to prevent interruptions in the infusion.

Syringe near empty alarms can be activated upon reaching thresholds of calculated remaining infusion time, or sensor-monitored volume remaining in the syringe. Both activation thresholds create issues for the user. First, a time-based threshold is accurate for continuous infusions that do not include boluses because the time remaining in an infusion can be easily calculated. However, a calculated time threshold is not accurate for infusions that include boluses because syringe pump control systems cannot predict when a bolus will be initiated by the patient and, therefore, the alarm is activated either very early or very late. Second, a volume-based threshold overcomes some of the issues of a time-based threshold. In that case, users set high volume thresholds for infusions that include PCA boluses. However, users must set low volume thresholds for continuous infusions.

Issues can occur if the user incorrectly programs a time-based threshold for an infusion that includes PCA boluses, or if the user sets a low volume threshold for an infusion that includes PCA boluses. There is also a high likelihood of human errors in the stressful hospital environment and, specifically, as the user interacts more with programming the pump control system in a medical setting.

A need accordingly exists for a syringe pump that minimizes user interaction with the syringe pump control system and automatically sets time- and/or volume-based activation thresholds for near empty syringe alarms based on user-inputted infusion therapy type.

### SUMMARY

Example systems, methods, and apparatus are disclosed herein for the automatic programming of a near empty alarm activation threshold based on an infusion therapy type selected by the user on a syringe pump. The example systems, methods, and apparatus are configured to use a preloaded drug library to determine if the selected infusion therapy type includes a PCA bolus or not, and define an appropriate time- or volume-based alarm activation threshold accordingly. The disclosed systems, methods, and apparatus prevent human programming errors by minimizing the need for extensive user interaction with syringe pumps. Further, the disclosed systems, methods, and apparatus allow for automatic programming to increase efficient infusion administration, and prevent programming errors that might result in patient discomfort or injury.

In light of the disclosure herein, and without limiting the scope of the invention in any way, in a first aspect of the present disclosure, which may be combined with any other aspect listed herein unless specified otherwise, a syringe pump includes an actuator, a syringe holder, a display screen, a memory, and a processor. The memory stores instructions that when executed by the processor, cause the processor to: receive a user-selected infusion therapy type, determine if the user-selected infusion therapy type selected includes PCA boluses or a continuous infusion without bolus, determine a volume-based threshold if the user-selected infusion therapy type includes PCA boluses, determine a time-based threshold if the infusion therapy type includes a continuous infusion without bolus, calculate or determine infusion therapy progress data related to an administration of an infusion therapy, the infusion therapy progress data including at least one of a total time of infusion or a remaining volume in the syringe, compare the total time of infusion to the time-based threshold when the user-selected infusion therapy type includes the continuous infusion without a bolus, compare the remaining volume in the syringe to the volume-based threshold when the user-selected infusion therapy type includes PCA boluses, and generate a message on the display screen displaying a user alert when the syringe holder on the syringe pump reaches the time-based or volume-based threshold.

In a second aspect of the present disclosure, which may be combined with any other aspect listed herein unless specified otherwise, a method of operating a syringe pump based on user input includes users selecting an infusion therapy type from a preloaded drug library displayed on a display screen, transmitting the infusion therapy type selected to a processor, the processor communicating with a memory and determining if the infusion therapy type selected includes PCA boluses or a continuous infusion without bolus, transmitting instructions from the memory to the processor that define a volume-based threshold if the infusion therapy type includes PCA boluses, transmitting instructions from the memory to the processor that define a time-based threshold if the infusion therapy type includes a continuous infusion without bolus, a control system automatically converting a user entered volume threshold to a time-based for a continuous infusion without bolus, generating a message on the display screen displaying a user alert when a syringe holder on the syringe pump reaches the time-based or volume-based threshold, and allowing users to control an actuator for the syringe pump based on user input.

In a third aspect of the present disclosure, which may be combined with any other aspect listed herein unless specified otherwise, the drug library includes information on whether the infusion therapy type is delivered as PCA boluses or a continuous infusion without bolus.

In a fourth aspect of the present disclosure, which may be combined with any other aspect listed herein unless specified otherwise, the user input is a volume-based threshold.

In a fifth aspect of the present disclosure, which may be combined with any other aspect listed herein unless specified otherwise, the user input is a time-based threshold.

In a sixth aspect of the present disclosure, which may be combined with any other aspect listed herein unless specified otherwise, the infusion therapy type is a medicine or supplement, the volume of a syringe, the syringe type, a time threshold, a volume threshold, and the volume of a bolus.

In a seventh aspect of the present disclosure, which may be combined with any other aspect listed herein unless specified otherwise, the preloaded drug library includes the name of an infusion therapy type.

In an eight aspect of the present disclosure, which may be combined with any other aspect listed herein unless specified otherwise, the memory matches the infusion therapy type selected from a preloaded drug library displayed on a display screen to an entry in the preloaded drug library, retrieves information on whether the infusion is delivered as PCA boluses or a continuous infusion without bolus.

In an ninth aspect of the present disclosure, which may be combined with any other aspect listed herein unless specified otherwise, the processor determines if the time-based threshold is reached by calculating a total infusion time based on a syringe volume and syringe type and an infusion rate, monitoring a running infusion time, and recognizing when the running infusion time is equivalent to the difference between the total infusion time and the time-based threshold.

In an tenth aspect of the present disclosure, which may be combined with any other aspect listed herein unless specified otherwise, the processor determines if the volume-based threshold is reached by calculating a total infusion volume using a syringe volume and syringe type, calculating a syringe volume displaced by a plunger using the syringe type, receiving data from a volume sensor tracking the position of a plunger in relation to a syringe, calculating a remaining syringe volume by comparing the difference between the total infusion volume and the syringe volume displaced, and recognizing when the remaining syringe volume equals the volume-based threshold.

In an eleventh aspect of the present disclosure, which may be combined with any other aspect listed herein unless specified otherwise, the processor determines if the volume-based threshold is reached by calculating a bolus administration time using the syringe volume, the syringe type, and a bolus volume, determining at the start of a bolus if the syringe volume after the bolus will be below the volume-based threshold, and calculating the specific time in the bolus administration time when the syringe volume will equal the volume-based threshold.

In a twelfth aspect of the present disclosure, which may be combined with any other aspect listed herein unless specified otherwise, transmitting a message to a clinician device.

In a thirteenth aspect of the present disclosure, which may be combined with any other aspect listed herein unless specified otherwise, the clinician device is one of a smartphone, a tablet computer, a laptop computer, a workstation, or a pager.

In a fourteenth aspect of the present disclosure, which may be combined with any other aspect listed herein unless specified otherwise, a syringe pump includes an actuator, a syringe holder, a display screen, a memory, and a processor. The memory stores instructions that when executed by the processor, cause the processor to: receive a user-selected infusion therapy type, determine whether the selected user-selected infusion therapy type includes PCA boluses or a continuous infusion without a bolus, determine whether the selected user-selected infusion therapy type includes a volume-based threshold or a time-based threshold, and generate a message on the display screen displaying a user alert when the syringe holder on the syringe pump reaches the time-based or volume-based threshold.

In a fifteenth aspect of the present disclosure, which may be combined with any other aspect listed herein unless specified otherwise, the processor determines a volume-based threshold when the user-selected infusion therapy type includes PCA boluses.

In a sixteenth aspect of the present disclosure, which may be combined with any other aspect listed herein unless specified otherwise, the processor calculates or determines a remaining volume in a syringe on the syringe holder.

In a seventeenth aspect of the present disclosure, which may be combined with any other aspect listed herein unless specified otherwise, the processor compares the remaining volume in the syringe to the volume-based threshold when the user-selected infusion therapy type includes PCA boluses.

In an eighteenth aspect of the present disclosure, which may be combined with any other aspect listed herein unless specified otherwise, the processor determines a time-based threshold when the user-selected infusion therapy type includes a continuous infusion without a bolus.

In a nineteenth aspect of the present disclosure, which may be combined with any other aspect listed herein unless specified otherwise, the processor calculates or determines total time of infusion.

In a twentieth aspect of the present disclosure, which may be combined with any other aspect listed herein unless specified otherwise, the processor compares the total time of infusion to the time-based threshold when the user-selected infusion therapy type includes the continuous infusion without a bolus.

In a twenty-first aspect of the present disclosure, any of the structure, functionality, and alternatives disclosed in connection with any one or more of FIGS. 1 to 5 may be combined with any other structure, functionality, and alternatives disclosed in connection with any other one or more of FIGS. 1 to 5.

In light of the present disclosure and the above aspects, it is therefore an advantage of the present disclosure to minimize user interaction with the syringe pump control system to minimize human error.

It is another advantage of the present disclosure to provide a syringe pump control system that automatically sets time and/or volume thresholds for near empty syringe alarms based on user-inputted infusion therapy type.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a system level diagram of a syringe pump within a hospital information system, according to an example embodiment of the present disclosure.

FIG. 2 is a perspective view of an example syringe pump comprising the Baxter® Novum pump, which may be included within the hospital system of FIG. 1, according to an example embodiment of the present disclosure.

FIG. 3 is a software component diagram of the operations performed by the syringe pump of FIG. 1, according to an example embodiment of the present disclosure.

FIG. 4 is a diagram of a pump control system process to activate a syringe near empty alarm based on user input, according to an example embodiment of the present disclosure.

FIG. 5 are sample views of a user interface for use in the display screen of the syringe pump of FIG. 1, according to an example embodiment of the present disclosure.

### DETAILED DESCRIPTION

Methods, systems, and apparatus are disclosed herein for a syringe pump that defines time- or volume-based near empty alarm activation thresholds based on a user-selected infusion therapy type (drug name, syringe volume, syringe type, and bolus volume when applicable). The example methods, systems, and apparatus are configured to automatically select a time- or volume-based alarm activation threshold after the user selects the drug being infused into a patient. As such, the user has minimal interaction with the syringe pump control system. Rather, the user selects the drug being infused and a processor and memory in the syringe pump communicate this information with each other. The processor matches the user-selected drug with an entry in a preloaded drug library. The entry from the preloaded drug library includes information on whether the drug's infusion protocol includes PCA boluses, or is continuous without PCA boluses. When the infusion protocol includes PCA boluses, the processor selects a volume-based near empty alarm activation threshold, finds the related instructions, and transmits them to the processor. Alternatively, when the infusion protocol does not include PCA boluses, the processor selects a time-based near empty alarm activation threshold, finds the related instructions, and transmits them to the processor. The processor executes the instructions it receives from the memory, monitoring the time or volume in the syringe pump, and activating the near empty alarm once the appropriate threshold is met. As such, there is no need for the user to decide whether to program a time- or volume-based threshold, as the syringe pump control system automatically programs it after the user selects the drug being infused.

Reference is made herein to a memory. As disclosed herein, a memory refers to a device that holds electronic data and/or instructions for immediate use by a processor and/or pump control system. The memory is able to receive and transmit data.

Reference is made herein to a processor. As disclosed herein, a processor refers to a device that executes instructions stored by the memory. The memory receives and transmits data.

Reference is made herein to an infusion. As disclosed herein, an infusion refers to the putting of fluids, intravenously through the use of a needle or catheter, into the bloodstream. The fluids can be a drug, supplement, or a mix thereof.

Reference is made herein to a drug library. As disclosed herein, a drug library refers to an indexed list of drugs and supplements. Each entry contains the name of the substance (both scientific and branded names), important parameters like maximum and minimum dosages, concentration information, infusion rates, and whether the drug is administered with or without PCA boluses. It should be noted that additional information can be included in the entries for a drug library.

While the example methods, apparatus, and systems are disclosed herein as operating with syringe pumps, it should be appreciated that the methods, apparatus, and systems may be operable with other pumps. For example, the methods, apparatus, and systems may provide for automatically setting near empty alarm thresholds in PCA pumps based on user-inputted infusion therapy type.

### Medical Environment Embodiment

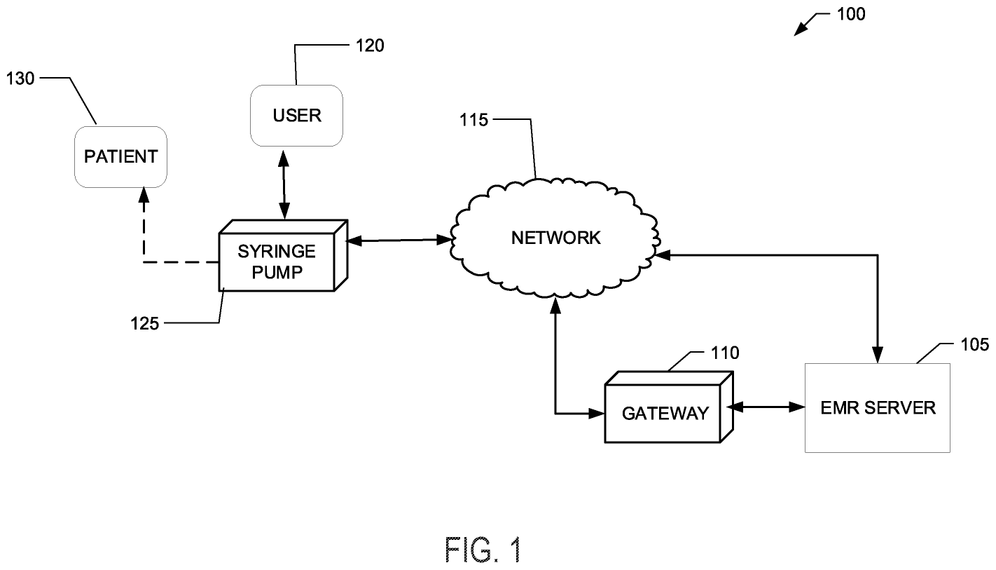

FIG. 1 is a system level diagram of a syringe pump within a hospital information system 100. The example system 100 includes a syringe pump 125, a network 115, a gateway 110, and an Electric Medical Records (“EMR”) server 105. The syringe pump 125 is capable of delivering an intravenous infusion therapy to a patient 130 via one or more intravenous (“IV”) line sets, based on inputs entered by the user 120. The syringe pump 125 connects to the network 115. In addition, the gateway 110 and EMR server 105 also connect to the network 115. As such, the syringe pump 125 is communicatively coupled to the gateway 110 and EMR server 105 via the network 115. In some embodiments, multiple syringe pumps 125 connect to the network 115, the gateway 110, and the EMR server 105. It must be noted that these connections may be wireless, such as via Bluetooth®, or wired, via a serial, Ethernet, CAN, or USB connection.

The gateway 110 is configured to receive infusion therapy type data (e.g., drug name, syringe volume, and bolus volume when applicable) from the syringe pump 125, and route the data to an EMR server 124. In some embodiments, the gateway 110 is configured to convert the data from, for example, EXTCOM message(s) to HL7 message(s). In yet other embodiments, the network 115 and the gateway 110 are omitted from the system 100.

The gateway 110 may also be configured to transmit operating parameters or prescription parameters to the syringe pump 125. For example, the gateway 110 may send an electronic prescription (or software update) to the syringe pump 125 at a predetermined time and/or when the syringe pump 125 is available to accept the prescription. In other instances, the syringe pump 125 may be configured to periodically poll the gateway 110 to determine if an electronic prescription (or software update) is awaiting to be downloaded to the pump.

Relatedly, the syringe pump 125 transmits infusion therapy progress data to the network 110. The network 110 then converts the therapy progress data to a protocol for transmission via Ethernet to the gateway 110 via the network 115. The gateway 110 may include, for example, the Baxter® IQ Enterprise® gateway. As such, the gateway 110 may be configured to integrate with the EMR server 105 or other hospital system to facilitate the transmission of the infusion therapy progress data from the syringe pump 125 to, for example, a hospital electronic medical record (“EMR”) related to the patient 130.

In one embodiment, the EMR server 105 is also communicatively coupled to a pharmacy server (not shown), which is configured to create and/or transmit medication orders corresponding to, for example, a prepared medication (not shown). A medication order includes an electronic record or entry, which identifies a patient (e.g., a patient identifier) and infusion parameters for administration. The medication order is assigned a unique identifier. In some embodiments, the medication order may be printed on a label attached to a medication container that is fluidly coupled to the syringe pump 125. The medication order itself associates a patient identifier with a medication identifier. The EMR server 124 is configured to use the patient identifier in the medication order to store or otherwise associate the medication order with a patient's EMR.

In an alternate embodiment, the system 100 may also include a clinician device (not shown; e.g., a smartphone, tablet computer, laptop computer, workstation, pager, etc.) such that the user 120 could monitor patient data.

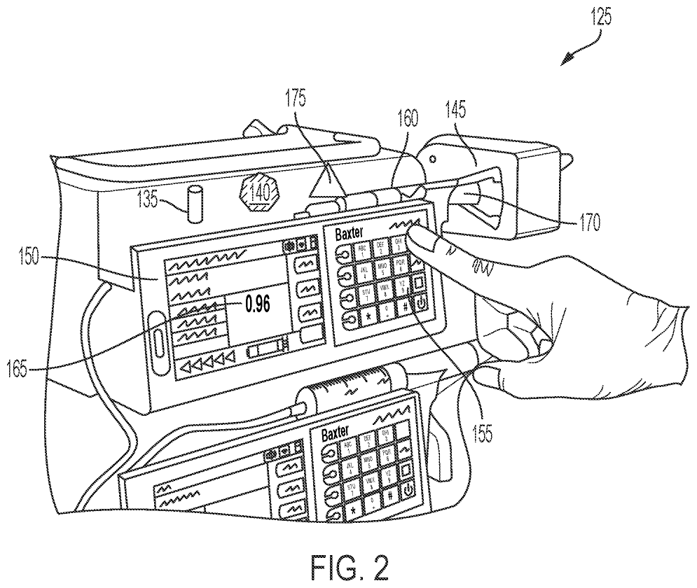

FIG. 2 is a perspective view of an example syringe pump 125. The illustrated infusion pump 125 is the Baxter® Novum IQ syringe pump. The syringe pump 125 uses a motor connected to an actuator 145 to actuate a plunger 170 within a syringe 160. In this embodiment, the syringe pump 125 includes a volume sensor 175 configured to monitor position of the plunger 170 in relation to the syringe 160. The illustrated syringe pump 125 includes a display 150 with interfaces 165 and a keypad 155 to enable a clinician to specify or program an infusion therapy.

The syringe pump 125 also includes a memory 135 and a processor 140. The memory 135 stores one or more drug libraries that include particular program parameter limits based on care area, dose change, rate of change, drug type, concentration, patient age, patient weight, etc. The limits are configured to ensure that a received prescription or entered infusion therapy is within acceptable ranges and/or limits decided by a medical facility, doctor, or clinician. The drug libraries also include information as to whether an infusion therapy includes PCA boluses, or administers at a continuous constant rate without PCA boluses.

The processor 140 is configured to execute machine-readable instructions stored in the memory 135. Execution of the machine-readable instructions by the processor 140 causes the syringe pump 125 to perform the operations described herein.

As noted previously, the syringe pump 125 is connected to and communicates with the gateway 110 (FIG. 1) via the network 115 (FIG. 1). As also noted, the syringe pump 125 is configured to monitor the progress of the infusion therapy and periodically transmit infusion therapy progress data (e.g., medical device data) to the gateway 110 (FIG. 1). The therapy progress data, as disclosed herein, may include, for example, an infusion rate, a dose, a total volume infused, a time remaining for the therapy, a medication concentration, rate change, a volume remaining within a medication container, a medication name, a patient identifier, titration information, bolus information, a care area identifier, a timestamp when the data was generated, an alarm condition, an alert condition, an event, etc. The syringe pump 125 may transmit the data continuously, periodically (e.g., every 30 seconds, 1 minute, etc.), or upon request by the gateway 110 (FIG. 1).

In some embodiments, the syringe pump 125 may also be communicatively coupled to one or more physiological sensors. For example, the syringe pump 125 may be connected to a pulse oximetry sensor, a blood pressure cuff, an access disconnection device, and/or a weight scale. The first pump 125 may be configured to integrate or otherwise include, for example, data from the pulse oximetry sensor into the therapy progress data or, alternatively, transmit the pulse oximetry data separately to the gateway 110 (FIG. 1). The gateway 110 (FIG. 1) can then access the EMR server 105 (FIG. 1) to record this data in a patient's electronic medical record.

### Setting Alarm Threshold

As previously noted, the near empty alarm can be activated based on a time-based or a volume-based activation threshold. If the user knows or wishes to define a time- or volume-based near empty alarm activation threshold, they can enter input it into the syringe pump 125 using the user interface (described below) and keypad 155. Alternatively, in this embodiment, the user can input the infusion therapy type (drug name, syringe volume, syringe type, and bolus volume when applicable) into the syringe pump 125 and the syringe pump 125 automatically defines the time- or volume-based threshold based on the drug.

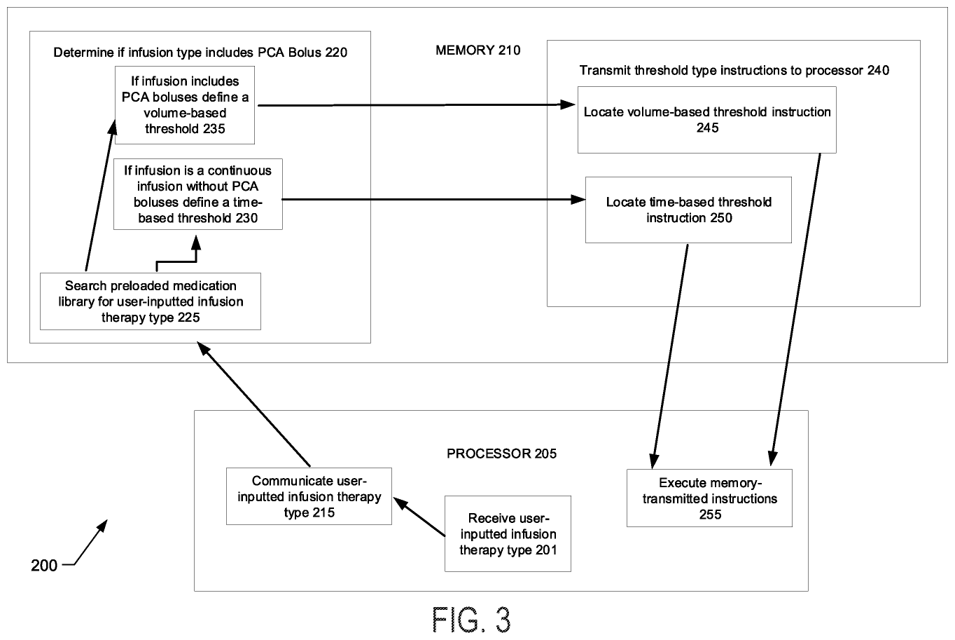

FIG. 3 is a software component diagram of the operations 200 performed by the syringe pump of FIG. 1 to define the near empty alarm threshold based on user input. As seen in the diagram, first the processor 205 receives the user-inputted infusion therapy type (drug name, syringe volume, syringe type, and bolus volume when applicable) data 201. The drug name is received in the form of a drug or supplement identifier. The processor 205 then transmits data containing the user-inputted drug name 215 to the memory 210. The memory 210 in conjunction with the processor 205 receives the drug name data and searches the drug library for a matching entry 225. More specifically, the memory 210 in conjunction with the processor 205 indexes its drug library, comparing the drug library entries to the drug or supplement identifier it received.

Upon finding a match, the processor 205 determines if the infusion protocol for the drug selected includes a PCA bolus 220. More specifically, the processor 205 accesses the matching drug library entry and retrieves data indicating if the infusion protocol includes any PCA boluses, or if it does not. If the infusion protocol includes a PCA bolus, then the processor 205 selects a volume-based threshold 235, and if it does not, it selects a time-based threshold 230. Once the processor 205 selects a volume- or time-based threshold, it locates either a volume-based threshold instruction 245, or it locates a time-based threshold instruction 250 in the memory 210. The memory 210 in conjunction with the processor 205 then transmits the instruction to the processor 240.

The processor 205 receives the memory-transmitted instructions and executes them. If the instructions are to implement a time-based threshold, then the processor monitors the infusion time and at the time threshold, sends an alert message to the display screen on the syringe pump and may set off an auditory alarm. More specifically, to implement a time-based threshold, the processor calculates the total infusion time using the syringe volume and syringe type inputted by the user, and the infusion rate it locates in the drug library. The processor monitors the infusion time throughout the infusion protocol and at the pre-determined time threshold, sends an alert message to the display screen on the syringe pump and may set off an auditory alarm. In an embodiment where the system includes a clinician device, the processor can also send an alert message to the clinician device. It should be noted that the specific time threshold (i.e. when 4 minutes remain in the infusion) can be determined by the user inputting it when they input the infusion therapy type, or it can be predefined in the drug library entry for the infusion therapy type selected by the user.

If the instructions are to implement a volume-based threshold, then the processor monitors the remaining volume in the syringe and at the volume threshold, sends an alert message to the display screen on the syringe pump and may set off an auditory alarm. More specifically, to implement a volume-based threshold, the processor calculates the total infusion volume using the syringe volume and syringe type inputted by the user. The processor also uses the syringe type to calculate the volume displaced by the movement of the plunger 170 (FIG. 2). Additionally, the processor calculates the time it takes to administer a bolus using the syringe volume, syringe type, and bolus volume inputted by the user. Throughout the infusion protocol, the processor monitors the remaining volume in the syringe 160 (FIG. 2) by consistently receiving data from the volume sensor 175 (FIG. 2) as a position of the plunger 170 (FIG. 2) in relation to the syringe 160 (FIG. 2), and calculates the difference between the initial drug volume and the drug volume administered by the movement of the plunger 170 (FIG. 2). When the syringe reaches the pre-determined volume threshold, the processor sends an alert message to the display screen on the syringe pump and may set off an auditory alarm. In an embodiment where the system includes a clinician device, the processor can also send an alert message to the clinician device. At the start of any bolus, the processor determines if the volume remaining post-bolus will be lower than the volume threshold. If the bolus will be lower than the volume threshold, the processor, using the time it takes to administers the bolus, calculates exactly when the syringe will reach the volume threshold, and sends the alert message at that point. It should be noted that the specific volume threshold (i.e. when 10 mL remain in the infusion) can be determined by the user inputting it when they input the infusion therapy type, or it can be predefined in the drug library entry for the infusion therapy type selected by the user.

It should also be noted that the user could also directly input a volume- or time-based threshold, rather than having the syringe pump control system define it based on entering an infusion therapy type (drug). However, if the user enters a volume-based threshold and the infusion protocol for the drug being administered is continuous without any PCA boluses, the control system will automatically convert the near empty alarm activation threshold to time-based.

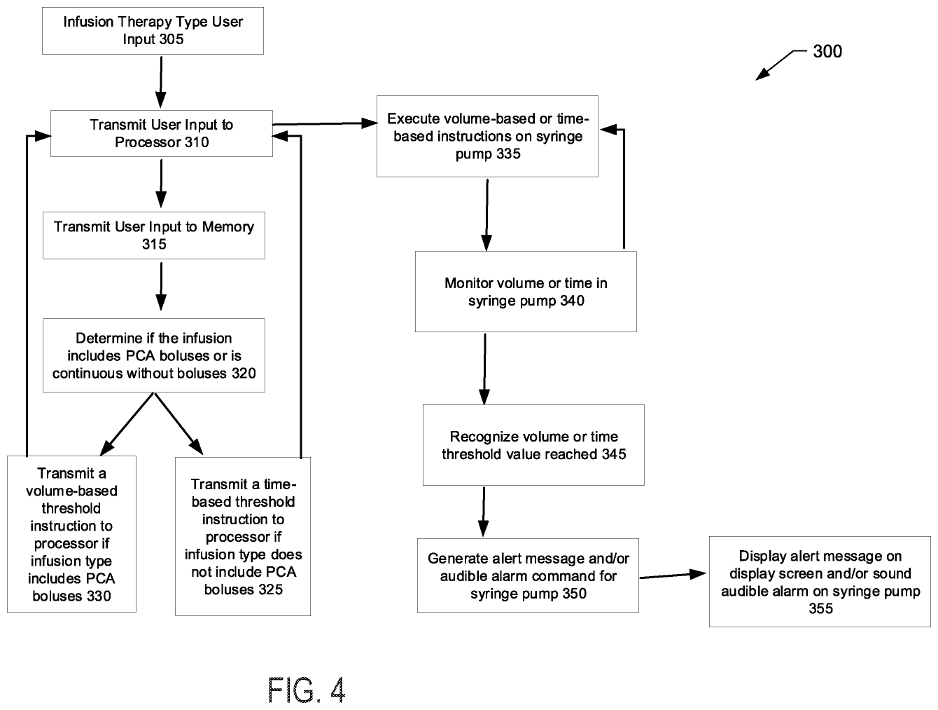

FIG. 4 shows a diagram of a pump control system process to activate a syringe near empty alarm based on user input. As seen in the diagram, the user inputs their desired infusion therapy type (block 305). The user does this by selecting, for example, a drug (an infusion therapy type) using a keypad and screen display on the syringe pump.

The data corresponding with the user input, a unique drug name, is then transmitted to the processor (block 310). Once it arrives at the processor, the processor transmits the user input data to the memory (block 315).

Upon arrival, the memory receives the user input data, locates the unique drug name identifier, and accesses its preloaded drug library to find a matching entry. Upon locating a match, the memory retrieves all data associated with the matching entry. The memory then determines if the matching entry, which corresponds with the drug (infusion therapy type) selected, has an infusion protocol that includes PCA boluses, or if it is continuous without PCA boluses. To achieve this, the memory retrieves, from the matching entry, the data indicating whether the infusion protocol includes any PCA boluses, or if it does not (block 320).

When the data indicates that the infusion protocol for the drug selected includes PCA boluses, the memory then retrieves instructions that define the near empty alarm activation threshold as volume-based, and transmits these instructions to the processor (block 330). On the other hand, when the data indicates that the infusion protocol for the drug selected does not include PCA boluses, the memory then retrieves instructions that define the near empty alarm activation threshold as time-based, and transmits these instructions to the processor (block 325).

The processor executes the volume- or time-based threshold instructions once it receives the instructions from the memory (block 335). More specifically, for a time-based threshold instruction, the processor calculates the time it will take to infuse the entire syringe at the instructed rate of infusion. The processor monitors the time remaining in the infusion by constantly communicating with the syringe pump (block 340). As such, the processor recognizes when the infusion reaches the time threshold (total time of infusion minus desired time for near empty alarm) (block 345). At this point, the processor generates an alert message and/or audible alarm command for the syringe pump (block 350). The processor then displays the alert message and/or audible alarm command and/or transmits the alert message to a server. The syringe pump subsequently displays the alert message on the display screen, and/or sounds the audible alarm on the syringe pump (block 355).

In the case of a volume-based threshold instruction, the processor monitors the volume in the syringe (block 340). The processor receives initial volume data from the volume sensor 175 (FIG. 2) related to the initial position of the plunger 170 (FIG. 2) in relation to the syringe 160 (FIG. 2). The processor assigns this initial volume data as equal to the entire volume of the infusion. Throughout the infusion, the processor continues to communicate with the volume sensor 175 (FIG. 2), calculating the remaining volume in the syringe as the difference in the initial volume and the equivalent volume of the change in movement of the plunger 170 (FIG. 2). The processor then recognizes when the infusion reaches the volume threshold (block 345). At this point, the processor generates an alert message and/or audible alarm command for display on the syringe pump (block 350). The processor may also transmit an alert message to a server. The syringe pump subsequently displays the alert message on the display screen, and/or sounds the audible alarm on the syringe pump (block 355).

### User Interface

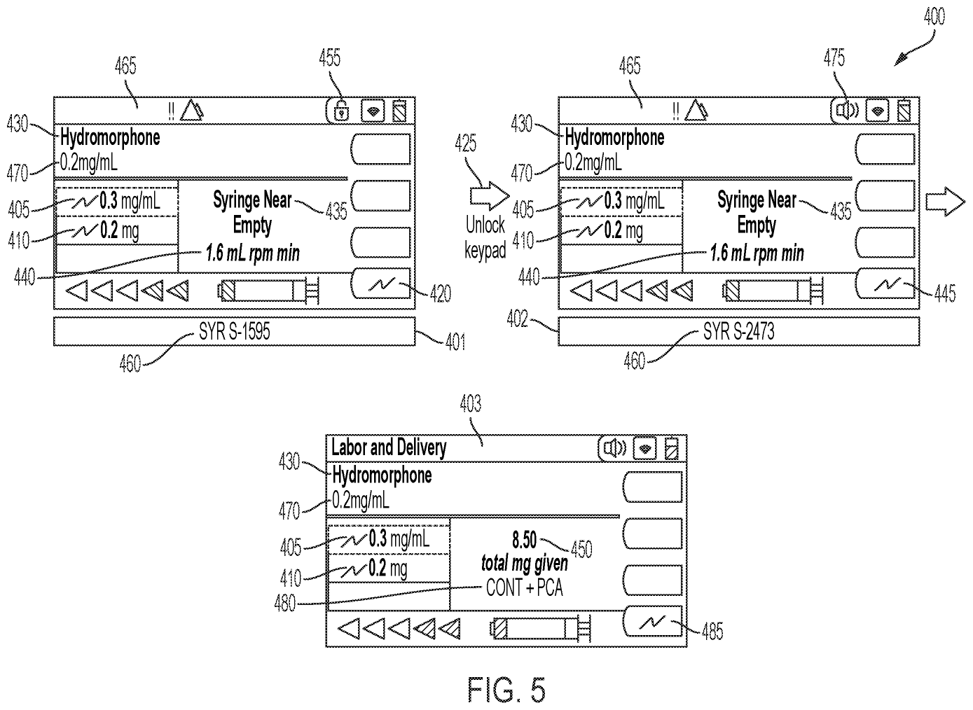

FIG. 5 shows diagrams of a user interface for use in the display screen of the syringe pump of FIG. 1, according to example embodiments of the present disclosure. As seen, the user interface has three different stages, locked 401, unlocked 402, and overview 403.

The locked interface 401 shows a “Syringe Near Empty,” or similar, alert message 435, a yellow banner 465 to get the user's attention, and an icon indicating the locked screen status 455. The interface 401 also shows the name of the drug infusion (infusion therapy type) 430 with the concentration 470 below it. The interface 401 also shows the remaining infusion volume 440, as well as the rate of continuous infusion 405 and bolus amounts 410, if applicable. In addition, a syringe identifier 460 is display on the interface 401. Finally, the interface 401 shows a silence digital button 420.

Once the user unlocks the keypad 425, the interface 401 changes to an unlocked interface 402 and the user can use the keypad to select the digital silence button 420. The unlocked interface 402 mirrors the locked interface 401, with two exceptions: it includes an alarm icon 475 in place of the locked icon 455, and it includes a digital dismiss button 445 that appears after the user uses the keypad to select the silence button 420. The user can use the keypad to select the dismiss button 445 to dismiss the alarm.

At this point, the user interface changes to the overview stage 403. The overview interface 403 shows the name of the drug infusion (infusion therapy type) 430 with the concentration 470 below it. The interface 403 also shows the rate of continuous infusion 405 and bolus amounts 410, if applicable. The interface 403 shows the total drug infusion administered 450 as well as the infusion protocol administered (i.e. continuous with PCA boluses) 480. Finally, the overview interface 403 shows a dismiss button 485, which the user can select using the keypad.

As such, the user interface provides the user with a comprehensive overview of the infusion protocol administered by the syringe pump. Moreover, the syringe pump disclosed herein provides various advantages to the user and the patient. The syringe pump minimizes user interaction with the syringe pump, thereby decreasing the chances of human errors that could lead to patient discomfort. Additionally, the syringe pump increases infusion efficiency and ease of use by automatically defining time- or volume-based near empty alarm activation thresholds based on the user's inputted infusion therapy type.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A syringe pump comprising:
an actuator;
a syringe holder;
a display screen;
a processor; and
a memory storing instructions, which when executed by the processor, cause the processor to
receive a user-selected infusion therapy type,
determine whether the selected user-selected infusion therapy type includes PCA boluses or a continuous infusion without a bolus,
determine a volume-based threshold when the user-selected infusion therapy type includes PCA boluses,
determine a time-based threshold when the user-selected infusion therapy type includes a continuous infusion without a bolus,
calculate or determine infusion therapy progress data related to an administration of an infusion therapy, the infusion therapy progress data including at least one of a total time of infusion or a remaining volume in a syringe,
compare the total time of infusion to the time-based threshold when the user-selected infusion therapy type includes the continuous infusion without a bolus,
compare the remaining volume in the syringe to the volume-based threshold when the user-selected infusion therapy type includes PCA boluses, and
generate a message on the display screen displaying a user alert when the syringe holder on the syringe pump reaches the time-based or volume-based threshold.

2. A method for operating a syringe pump based on user input comprising:
selecting an infusion therapy type from a preloaded drug library displayed on a display screen;
transmitting the infusion therapy type selected to a processor, wherein the processor communicates with a memory and determines if the infusion therapy type selected includes PCA boluses or a continuous infusion without bolus;
transmitting instructions from the memory to the processor that define a volume-based threshold if the infusion therapy type includes PCA boluses;
transmitting instructions from the memory to the processor that define a time-based threshold if the infusion therapy type includes a continuous infusion without bolus, wherein a control system will automatically convert a user entered volume threshold to a time-based for a continuous infusion without bolus;
generating a message on the display screen displaying a user alert when a syringe holder on the syringe pump reaches the time-based or volume-based threshold; and
allowing users to control an actuator for the syringe pump based on user input.

3. The method of claim 2, wherein the preloaded drug library includes information on whether the infusion therapy type is delivered as PCA boluses or a continuous infusion without bolus.

4. The method of claim 2, wherein the user input is a volume-based threshold.

5. The method of claim 2, wherein the user input is a time-based threshold.

6. The method of claim 2, wherein the infusion therapy type is a medicine or supplement, the volume of a syringe, the syringe type, a time threshold, a volume threshold, and the volume of a bolus.

7. The method of claim 2, wherein the preloaded drug library includes the name of an infusion therapy type.

8. The method of claim 6, wherein the memory matches the infusion therapy type selected from a preloaded drug library displayed on a display screen to an entry in the preloaded drug library, retrieves information on whether the infusion is delivered as PCA boluses or a continuous infusion without bolus.

9. The method of claim 2, wherein the processor determines if the time-based threshold is reached by calculating a total infusion time based on a syringe volume and syringe type and an infusion rate, monitoring a running infusion time, and recognizing when the running infusion time is equivalent to the difference between the total infusion time and the time-based threshold.

10. The method of claim 2, wherein the processor determines if the volume-based threshold is reached by calculating a total infusion volume using a syringe volume and syringe type, calculating a syringe volume displaced by a plunger using the syringe type, receiving data from a volume sensor tracking the position of a plunger in relation to a syringe, calculating a remaining syringe volume by comparing the difference between the total infusion volume and the syringe volume displaced, and recognizing when the remaining syringe volume equals the volume-based threshold.

11. The method of claim 11, wherein the processor determines if the volume-based threshold is reached by calculating a bolus administration time using the syringe volume, the syringe type, and a bolus volume, determining at the start of a bolus if the syringe volume after the bolus will be below the volume-based threshold, and calculating the specific time in the bolus administration time when the syringe volume will equal the volume-based threshold.

12. The method of claim 11, further comprising transmitting a message to a clinician device.

13. The method of claim 12, wherein the clinician device is one of a smartphone, a tablet computer, a laptop computer, a workstation, or a pager.

14. A syringe pump comprising:
an actuator;
a syringe holder;
a display screen;
a processor; and
a memory storing instructions, which when executed by the processor, cause the processor to
receive a user-selected infusion therapy type,
determine whether the selected user-selected infusion therapy type includes PCA boluses or a continuous infusion without a bolus,
determine whether the selected user-selected infusion therapy type includes a volume-based threshold or a time-based threshold, and
generate a message on the display screen displaying a user alert when the syringe holder on the syringe pump reaches the time-based or volume-based threshold.

15. The syringe pump of claim 14, wherein the processor determines a volume-based threshold when the user-selected infusion therapy type includes PCA boluses.

16. The syringe pump of claim 15, wherein the processor calculates or determines a remaining volume in a syringe on the syringe holder.

17. The syringe pump of claim 16, wherein the processor compares the remaining volume in the syringe to the volume-based threshold when the user-selected infusion therapy type includes PCA boluses.

18. The syringe pump of claim 14, wherein the processor determines a time-based threshold when the user-selected infusion therapy type includes a continuous infusion without a bolus.

19. The syringe pump of claim 18, wherein the processor calculates or determines total time of infusion.

20. The syringe pump of claim 19, wherein the processor compares the total time of infusion to the time-based threshold when the user-selected infusion therapy type includes the continuous infusion without a bolus.
